(19) United States
(12) Plant Patent Application Publication
(10) Pub. No.: US 2015/0282413 P1
(43) Pub. Date: Oct. 1, 2015

(54) Adenanthos plant named 'Tal'

(50) ;

(71) Applicant: Nitzan Zir, Kfar Hess (IL)

(72) Inventor: Nitzan Zir, Kfar Hess (IL)

(21) Appl. No.: 13/999,888

(22) Filed: Mar. 31, 2014

(51) Int. Cl. A01H 5/00

(52) U.S. Cl. PLT/373

(57) ABSTRACT

A new and distinct Adenanthos cultivar named ‘TAL’ is disclosed, characterized by unique branch structure. The new variety is a Adenanthos, normally used for ornamental foliage, or landscape purposes.

### LATIN NAME OF THE GENUS AND SPECIES

Adenanthos sericeus

### VARIETY DENOMINATION

‘TAL’

### BACKGROUND OF THE INVENTION

The new Adenanthos cultivar is a product of open pollination. The new variety was discovered as a seedling growing in a field of various Adenanthos sericeus varieties by the inventor, Nitzan Nir, in Kfar Hess, Israel. The new variety was selected during 2008, at a commercial nursery in Kfar Hess, Israel. The exact parents are unknown, and believed to be unnamed varieties of Adenanthos.

Asexual reproduction of the new cultivar has been performed by vegetative cuttings. This was first performed at a commercial nursery in Kfar Hess, Israel in 2008 and has shown that the unique features of this cultivar are stable and reproduced true to type in 6 successive generations.

### SUMMARY OF THE INVENTION

The cultivar ‘TAL’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘TAL’. These characteristics in combination distinguish ‘TAL’ as a new and distinct Adenanthos cultivar:

1. Unique branch structure.

### PARENT COMPARISON

The parents are unknown to the inventor.

### COMMERCIAL COMPARISON

Plants of the new cultivar ‘TAL’ are compared to the unnamed, unpatented commercial variety Adenanthos sericeus. Varieties are similar in most horticultural characteristics; however ‘AVIV’ differs in the following characteristics;

1. Different branch structure.

Plants of the new cultivar ‘TAL’ can also be compared to the commercial variety Adenanthos ‘AVIV’; filed concurrently. The varieties are similar in most horticultural characteristics; however ‘TAL’ differs in the following characteristics;

1. Different branch structure

2. Upright plant habit, compared to prostrate plant habit.

### BRIEF DESCRIPTION OF THE PHOTOGRAPH

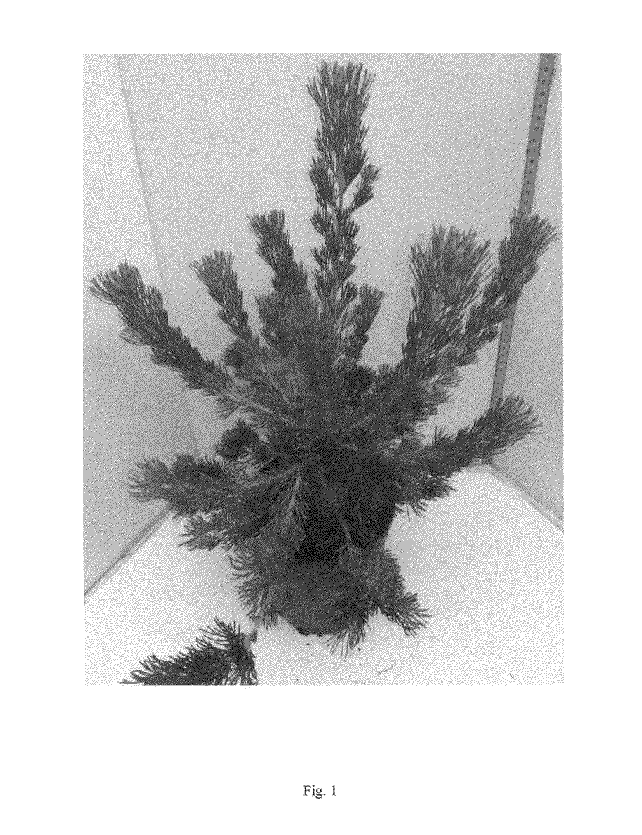

The accompanying photograph in FIG. 1 illustrates in full color a typical plant of ‘TAL’ grown in a commercial greenhouse in Kfar, Israel. The plant is 4-5 months old from a rooted cutting, in a 15 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe typical plant of ‘TAL’ grown in a commercial greenhouse in Kfar, Israel. The plant is at least 2 months old from a rooted cutting, in a 25 cm pot. The growing temperature ranged from 15° C. to 35° C. during the day and from −2° C. to 35° C. during the night. General light conditions are bright, normal sunlight. Measurements and numerical values represent averages of typical plant types. Chemical treatments included general fungicides and insecticides.
Botanical classification: Adenanthos sericeus ‘TAL’
Propagation:
   Time to produce a rooted plantlet.—30 to 60 days at approximately 24° C.
   Root description.—Split root.
Plant:
   Growth habit.—.
   Height.—300-500 cm to top of foliage.
   Plant spread.—200 cm.
   Growth rate.—.
   Branching characteristics.—.
   Length of primary lateral branches.—60 cm on 4 to 5 month plant.
   Quantity of primary lateral branches.—120.
   Characteristics of primary lateral branches.—Form: Round Diameter: 0.4-0.6 cm. Color: Greyed-green 194A RHS Texture: Hairy Strength: Flexible.
   Internode length.—0.5-7.0 cm. (measured in section of 20 cm from top).
Foliage:
   Leaf.—Arrangement: Alternate Quantity: Approximately 9 leaves, in the 10-15 cm section from the top of the branch. Average Length: 3.7 cm. Average Width: 2.0 cm. Shape of blade: Needle Apex: Needle-shaped Base: Elliptical Margin: Round Texture of surface: Hairy Aspect: Straight Color: Young foliage upper side: Yellow-green 146C RHS Young foliage under side: Yellow-green 146C RHS Mature foliage upper side: Green 137A RHS Mature foliage under side: Green 137A RHS Venation: Indistinguishable. Petiole: Length: 0.6 cm. Diameter: 0.1 cm. Color: Green 137A RHS Texture: Hairy.
Flower:
   Natural flowering season.—Flowering rare in Adenanthos and not observed to date with new variety.
Other characteristics:
   Disease/pest resistance.—Neither resistance nor susceptibility to the normal diseases and pests of Adenanthos have been observed to date.
   Temperature tolerance.—From −2° C. to 35° C.
   Drought tolerance.—Extremely drought tolerant.

What is claimed is:

1. A new and distinct cultivar of Adenanthos plant named ‘TAL’ as herein illustrated and described.
